(19) United States
(12) Patent Application Publication
(10) Pub. No.: US 2023/0218791 A1
(43) Pub. Date: Jul. 13, 2023

(54) LAMP AND SYSTEM WITH WALL-TYPE RADIATION FIELDS FOR PREVENTING OR MINIMISING THE SPREAD OF PATHOGENS IN INDOOR AIR

(71) Applicant: SMART UNITED HOLDING GMBH, Gruenwald (DE)

(72) Inventors: Reiner PROHASKA, Gruenwald (DE); Andreas WIESER, Muenchen (DE); Julius MUSCHAWECK, Gauting (DE)

(21) Appl. No.: 18/009,263

(22) PCT Filed: Jun. 11, 2021
(86) PCT No.: PCT/EP2021/000073

§ 371 Date: Dec. 8, 2022

(30) Foreign Application Priority Data
Jun. 11, 2020 (EP) 20 179 592.9
Aug. 26, 2020 (DE) 10 2020 122 343.9
Sep. 29, 2020 (DE) 10 2020 125 384.2
Nov. 20, 2020 (EP) 20 209 035.3
Jan. 28, 2021 (EP) 21 000 027.9

(51) Int. Cl. A61L 2/10; A61L 2/26; A61L 2/24

(52) U.S. Cl. CPC A61L 2/10; A61L 2/26; A61L 2/24; A61L 2202/11; A61L 2202/25; A61L 2202/14

(57) ABSTRACT

The invention is directed to a system for preventing or minimizing the spread of viruses, and to the prevention or minimization of the spread of viruses in indoor air, including one or more radiation sources (10) in a room which divide the room into smaller segments using UV-C light walls, a sensor system for detecting a movement or a presence of one or more persons (P) in the room, and a controller (16) that is designed to at least partially switch the one or more radiation sources (10) on or off as a function of at least the presence of the person (P).

According to the invention, the one or more radiation sources (10) are designed to generate a wall-type radiation field (10b) that acts as a UV-C wall, so that the room or rooms is/are divided into smaller room segments, which prevents or minimizes the spread of viruses due to the fact that the viruses are deactivated by the UV-C light, and the controller (16) is designed to at least partially switch off the radiation source (10) in question if the movement data detected by the sensor system indicate a likelihood that one of the persons (P) would like to pass through the radiation field (10b) in question.

The invention relates to a lamp and a system with wall-like radiation fields for disinfecting indoor air, and to the prevention/minimization of the spread of pathogens, in particular viruses, in buildings, and a corresponding method.

Even well before the worldwide coronavirus pandemic, the health significance of hygienic measures in rooms, in particular where numerous persons work together and/or enter and exit, has been known.

In this regard, various systems for disinfecting indoor air using UV radiation have already been proposed.

UV radiation is appropriate germicidal radiation. A suitable radiation source is therefore a UV lamp, which generally emits UV radiation approximately in the wavelength range of 100 to 400 nm. Within the UV radiation, the germicidal effect increases from UV-A to UV-B to UV-C, as the wavelength becomes shorter. Thus, a UV-C lamp that specifically emits UV-C radiation approximately in the wavelength range of 100 to 280 nm is particularly suitable. The wavelength range of approximately 200 to 280 nm is preferred, since in this range, air is essentially transparent to the radiation. Known radiation sources of this type are mercury vapor lamps or light-emitting diodes or laser diodes for emitting appropriate UV light.

Germicidal UV-C radiation may be harmful to the human eyes and skin. Measures for thus protecting persons from exposure to the germicidal UV-C radiation may include a mirror, a screen, and/or a shield for bundling, directing, and limiting the radiation. These measures preferably include a sensor for detecting the presence of a person in the spatial area in which the radiation may act, in particular for detecting the presence of a person in the area immediately in front of or next to the radiation source. A switch is coupled to the sensor and the radiation source, and switches them off when the sensor detects the person.

However, in the paper by Welch, D., Buonanno, M., Grilj, V. et al., “Far UV-C light: A new tool to control the spread of airborne-mediated microbial diseases,” Sci Rep 8, 2752 (2018), it is described that very short-wave UV-C light (207-222 nm), also referred to as far UV-C light, efficiently inactivates bacteria without damaging the exposed skin of mammals. The reason is that far UV-C light, due to its strong absorption in biological materials, is not able to penetrate the outer (nonliving) layers of the human skin or of the eye. However, since bacteria and viruses have dimensions of 1 micron or less, far UV-C light can penetrate and inactivate them. It has been shown that far UV-C efficiently inactivates aerosolized viruses in the air; a very low dose of 2 mJ/cm² of 222 nm light inactivates more than 95% of an aerosolized H1N1 influenza virus.

Suitable sensors for detecting the presence of persons are motion detectors such as ultrasonic or radar sensors, which make use of the Doppler effect when their emitted ultrasonic or radar radiation is reflected on a moving person, or passive pyroelectric IR (PIR) sensors, which detect the changes in thermal radiation in the surroundings of furniture, caused by a moving person. Also suitable are proximity sensors such as capacitive, optical, ultrasonic, or radar sensors, which can detect a person in the vicinity, regardless of his/her movement.

A system for decontaminating wet rooms in hospitals is known from WO 2016/049143 A1, for example. Situated in the wet room is a UV-C light source that is switched off as soon as a person enters the room.

A system including UV radiation sources for installation in passenger cabins of aircraft is known from U.S. Pat. No. 9,550,006 B2. A safety system activates or deactivates the radiation sources when a passenger or crew member enters the passenger cabin.

A mobile system for decontaminating hospital rooms is known from U.S. Pat. No. 9,095,633 B1. This system is set up in the hospital room, and is started via a timer switch when all persons have left the room.

UV-impermeable radiation protection curtains are known from WO 2015/054389 A2, with which certain areas of a room to be disinfected (for example, a bed in a multibed hospital room) may be partitioned off to allow disinfection of the partitioned-off area using UV radiation sources, while persons in other areas of the room may remain. A similar system for the same purpose is known from WO 2014/100493 A1, having partition walls with UV radiation sources situated at their inner side.

A mobile UV radiation source that may be set up in rooms to be disinfected or in room areas that are partitioned off with UV protection is known from WO 2012/142427 A1 or U.S. Pat. No. 6,656,424 B1, for example.

One disadvantage of the above-mentioned systems is that either persons must be completely absent from the room to be decontaminated, or a complicated structure of radiation protection walls or curtains is necessary. This is not achievable in rooms with frequent and unpredictable use by the public.

For continuous disinfection of indoor air in rooms that are continually occupied, for example waiting rooms of doctors' offices, it is known to situate UV-C radiation sources inside housings of fans, air conditioners, or ventilators. For example, such a device for mounting on the ceiling is known from US 2009 004046 A1. One disadvantage of these systems that operate using a circulating air process is that diffusion of possibly infectious aerosols between persons present in the room in question remains possible, and due to the airflow of the circulating air, the aerosols may spread even more quickly than would be the case without the air circulation system.

A device for sterilizing air in a closed room is also known from KR 102152810 B1. A UV light-emitting tube whose radiated light is to be formed into the most parallel light beam possible with the aid of optical devices is used as the light source. During operation, the lamp is oriented in such a way that it emits the UV light in the direction of the ceiling or an upper wall section if persons are in the room. To allow a larger volume of air to be sterilized in the room, the lamp may be rotated if no persons who could be damaged by the UV radiation are in the room. However, due to the widening of the emitted radiation, the lamp can be used only for sterilizing areas that are completely free of persons. This is ensured when the irradiated area is so high that persons typically are not present there. The dimensions of the generated radiation field do not allow operation between persons in order to prevent transport of viruses, or pathogens in general, at that location from one person to another.

The object of the invention is to provide a lamp and a system with wall-like radiation fields, generated by one or more such lamps, for preventing or minimizing the spread of pathogens in indoor air, which, when the radiation fields are positioned between persons, effectively prevent transmission of pathogens between these persons without impairing their freedom of movement.

The object is achieved by a lamp for forming a barrier in the form of a wall-like radiation field having the features of claim 1, and a system with such wall-like radiation fields for preventing or minimizing the spread of pathogens, in particular viruses, in indoor air. Advantageous embodiments of the invention result from the subclaims. Within the meaning of the present discussion, “barrier” is not to be understood in the sense of a mechanical boundary. Rather, “barrier” means that although pathogens, in particular viruses, may pass to the other side of the barrier, they are deactivated upon passing through the barrier.

The invention relates to a lamp and to a system for preventing or minimizing the spread of pathogens in indoor air, including one or more such lamps as radiation sources in a room or multiple rooms, in particular including a sensor system for detecting a movement or a presence of one or more persons in the room, and a controller that is designed to switch the one or more radiation sources on or off as a function of at least the presence of the person.

For forming the barrier for pathogens, the lamp for achieving the germicidal effect includes at least one illuminant that emits UV-C radiation. In the following discussion, for simplification, reference is usually made solely to viruses as an example, although the barrier designed according to the invention is also effective with regard to bacteria. The radiation emitted by the one or more illuminants is collimated with the aid of an optical device, resulting in a radiation field whose thickness is at least one order of magnitude smaller than its length and width, i.e., is at most only 1/10 of the length or width.

The lamp includes a plurality of illuminants that emit UV-C radiation. LEDs are preferred illuminants. Compared to the tubes used in the prior art, LEDs have the advantage that they are available with narrow bandwidths, so that LEDs that imitate [sic; emit] radiation with a wavelength above 242 nm may be selected for the lamp. This ensures that the generated UV light causes little or no formation of ozone. The lamps are thus particularly suitable for use in rooms in which persons are present. Irritation from ozone resulting from the sterilization is thus avoided. In addition, the optical device of the lamp includes a plurality of optical elements for collimating the radiation emitted by the illuminants. In each case at least one optical element is associated with each illuminant. The optical elements associated with the particular illuminant are designed in such a way that the dimensions of the radiation emitted by the illuminant and by the optical element(s) in a direction perpendicular to the radiation direction is less than 12 cm, in particular less than 8 cm, particularly preferably less than 5 cm. Arranging multiple such illuminants, together with their associated optical elements, in a consecutive series thus allows formation of the radiation wall having the dimensions described above.

The optical device is thus designed in such a way that the radiation emitted by the illuminant exits essentially only within a region that is bounded by two mutually parallel planes. The distance between these planes is the above-mentioned thickness. The “length” refers to the dimensions in the direction of the radiation exiting from the lamp, and the “width” refers to an extension perpendicular to the length and the thickness. The length is understood as an at least utilizable extension of the radiation field in the radiation direction, which for an installation of the lamp on the ceiling of a room, for example, is the distance to the floor. Typical dimensions of rooms have heights of up to 5 m, so that an at least utilizable extension of 5 m is preferably to be provided. It is preferred that the radiation emitted by the illuminant be collimated even more intensely, so that the thickness is preferably at least two orders of magnitude smaller than the minimum length and width of the radiation field.

For a lamp that is to be used in a room, it is particularly preferred that the thickness, i.e., the distance between the parallel planes in which the collimated radiation propagates, for an at least utilizable extension of 5 m does not exceed a value of 8 cm, preferably 5 cm. Typical widths of the radiation field, and thus also of the lamps, may likewise be up to 5 m. However, it is preferred that the lamps have a shorter length, which significantly simplifies installation and transport. To achieve greater overall widths, the lamps may then also be successively arranged in a line.

The optical device may preferably also include a screening device. This screening device prevents radiation components from laterally exiting from the radiation field. The screening device may be formed by a plurality of channels, for example; the totality of the channels forms a light exit surface, or is situated in front of the light exit surface in the lamp, and all radiation that is emitted by the lamp can exit only through this totality of channels. The walls of the channels are coated with material that absorbs the emitted radiation or are made of absorbent material. In this way, only the collimated portion of the radiation emitted by the illuminants can exit through the channels without absorption. Scattered light that corresponds to the uncollimated portion of the radiation is hindered by the screening device at the exit to the surroundings. The UV-C radiation ultimately leaving the lamp may thus be efficiently restricted to the region that is formed between the bounding imaginary planes. This region forms a so-called UV wall.

The plurality of illuminants and their associated optical elements form at least one group. The radiation directions that result for each illuminant due to the at least one associated optical element, or the radiation directions of assemblies of a group, are parallel to one another for all illuminants or assemblies that belong to the same group, and are situated in a shared surface area, in particular a plane. An assembly in each case includes multiple illuminants and the corresponding associated elements within a group. A plurality of individual illuminants may thus cooperate with the particular optical element associated with the illuminant in each case to form the wall-like radiation field described above. For forming two or more groups, the groups may in particular be designed in such a way that the radiation directions of the illuminants of one group are oriented in parallel to the radiation directions of the illuminants of the other group.

It is particularly preferred that the optical elements are designed and situated in the lamp in such a way that the collimated radiation of one illuminant overlaps or at least directly adjoins the collimated radiation of a neighboring illuminant of the same group. In this way, the radiation field of the totality of illuminants associated in each case with a group jointly forms a seamless barrier, also referred to as a wall-like radiation field or UV wall. It is also conceivable to arrange multiple such groups in parallel to one another with identical distances between the individual illuminants, and to arrange the groups offset relative to one another in the longitudinal direction of the lamp. However, the shift in the longitudinal direction is less than the distance, ideally one-half the distance, between successive illuminants. The combination of multiple groups thus results in a shared, continuous radiation field in the longitudinal direction of the lamp, even if no seamless radiation field results within a group.

Each group of illuminants together with their optical elements may also be divided into subgroups, and independently switching the illuminants of various subgroups on and off may be made possible. Thus, for example, only a small portion of illuminants from a group that collectively forms a relatively large barrier for viruses may be switched off if a safety risk for a person could arise in this area. The other subgroups, meanwhile, may remain switched on. Not switching off all illuminants of the entire lamp has the advantage that the radiation field may be maintained, at least in partial areas, so that at least partial protection remains. The granularity may be determined based on how many subgroups a group of illuminants is divided into. In the extreme case, in each case one illuminant forms a subgroup.

It is also proposed that the one or more radiation sources, which may be formed by the lamps described above, are designed in each case to generate via bundled UV-C light a wall-like radiation field that acts as a UV-C wall, in order to divide the room or rooms into smaller room segments and prevent or minimize the spread of viruses, since the viruses are deactivated by the UV-C light. In variants that include a sensor system, the controller is advantageously designed to switch off the radiation source in question or portions thereof when the movement data, detected by the sensor system(s), show that one of the persons is approaching the radiation field in question.

Such an approach to the radiation field may be assumed, for example, when the sensor system determines a penetration of an object into a safety zone that is adjacent to the radiation field and monitored by the sensor system. The penetration by an object may involve a person (or only a body part of the person, such as a finger), as well as other objects. Due to the detection of the penetration of objects into the safety area and appropriate (selective) switching off of the appropriate subgroup or subgroups of illuminants, indirect endangerment of persons from reflected radiation components may also be prevented from arising. For selectively switching off one or more subgroups, the penetration of the objects into the safety zone is determined in at least one dimension, with spatial resolution.

In particular when far UV-C radiation is used, for the above-described reasons the sensor system and the switching off may also be dispensed with, since there is no concern for health risks.

The wall-like radiation fields, which in particular contain high-intensity UV-C radiation, form diffusion barriers for pathogens. The intensity and wavelength of the radiation field are coordinated in such a way that pathogens or viruses that may be contained in aerosols or droplets are killed upon passing through the wall-like radiation field. The likelihood of infection for persons present in room segments that are separated from one another by such a radiation field may thus be greatly reduced. Even if the viruses are not completely killed, an effect may be achieved that corresponds to or is superior to that of protective face masks or “social distancing” measures.

An algorithm that computes the likelihood of an imminent traversal through the radiation field may take into account not only the position of the person, i.e., the proximity of the person to the radiation field, but also the direction of movement and the speed of movement of the person, as well as certain boundary conditions of the room. Such boundary conditions may be defined, for example, by items of furniture whose locations are stored in the controller. Under normal circumstances, it is very unlikely that a person would climb or jump over a table or a room divider.

A “wall-like” radiation field refers to a radiation field that forms an approximately two-dimensional surface, i.e., has a thickness that is at least one order of magnitude smaller than its length and its width. The wall-like radiation field may in particular also be made up of multiple closely spaced beams, for example laser beams, oriented in parallel.

The invention is applicable to various rooms in which humans are present, for example open-plan offices, classrooms in schools, multiple-bed hospital rooms, restaurants, or workstations in industry.

Due to the advantages discussed above in conjunction with the paper by Welch et al., embodiments are particularly advantageous in which the virus-deactivating UV-C radiation is far UV-C radiation having a wavelength in the range of 200-222 nm, in particular 207-222 nm. Due to the technically highly developed radiation sources and for cost reasons, in certain fields of application even a wavelength range of 223-280 nm may be advantageous.

Furthermore, it is proposed that the one or more radiation sources are designed as light strips for ceiling or wall mounting. Each of the radiation sources may be equipped with one or more UV-C radiators, for example LEDs or laser diodes, or may include a stronger UV light source such as a mercury vapor lamp or a pumped laser, whose light may then be split in a fan-like manner by use of a suitable optical arrangement in order to generate the desired wall-like shape. Flexible use, also for the retrofitting of rooms, is possible due to the design as mountable light strips.

When the system includes freely movable stands for holding one or more radiation sources, the system is also usable in areas in which the spatial conditions do not allow wall or ceiling mounting.

In a further embodiment of the invention, the one or more radiation sources are each designed to generate multiple parallel radiation fields, so that a double wall or multiple wall is created. The protective effect may thus be further enhanced.

Moreover, it is proposed that the one or more radiation sources are designed for arrangement along boundaries of room segments, the controller being designed to activate the radiation sources in question when one or more persons are present in the particular room segment, and to deactivate at least one of the radiation sources when a person enters or leaves the room segment.

In addition, it is proposed that the room segments form a regular grid. Large-area rooms may thus be flexibly covered.

In a further embodiment of the invention, further radiation sources having a disinfecting or virus-deactivating effect are situated in the room segments. The controller may then be designed to activate the further radiation sources when no person is present in the room segment. In this way, surfaces, computers, chairs, etc. may be effectively disinfected when no person is present in the room segment.

Furthermore, it is proposed that the sensor system includes a 3D camera or TOF camera and/or one or more CCD cameras to allow the three-dimensional position and pose of the persons in the room segment in question to be detected and evaluated.

A further aspect of the invention relates to a method for preventing or minimizing the spread of viruses in indoor air, using one or more radiation sources in a room, optionally including detecting a movement or a presence of one or more persons in the room and automatically switching one or more radiation sources on or off as a function of at least the presence of the person.

It is proposed that the one or more radiation sources are designed in each case to generate a wall-like radiation field that acts as a UV-C wall, in order to divide the room or the rooms into smaller room segments that prevent or minimize the spread of viruses, since the viruses are deactivated by the UV-C light, and the method includes switching off the radiation source in question when the movement data detected by the sensor system indicate that it is likely that one of the persons would like to pass through the radiation field in question.

The invention further relates to a system for preventing or minimizing the spread of viruses in rooms and for disinfecting indoor air using one or more interconnected radiation sources, characterized in that the one or more interconnected radiation sources form so-called light walls due to bundled UV-C light, and thus divide rooms into smaller segments that prevent or minimize the spread of viruses, since the viruses are deactivated by the UV-C light, in combination with motion detectors that switch off the individual UV-C light walls when a person approaches, or switch them back on when the person leaves, and an additional UV-C radiator that irradiates the individual parcels resulting from the one or more UV-C light walls and deactivates the aerosols (viruses present in the air).

Further features and advantages result from the following description of the figures. The entire description, the claims, and the figures disclose features of the invention in specific exemplary embodiments and combinations. One skilled in the art will also consider the features individually and combine them into further combinations or subcombinations in order to adapt the invention, as defined in the claims, to the needs or specific areas of application.

In the drawings:

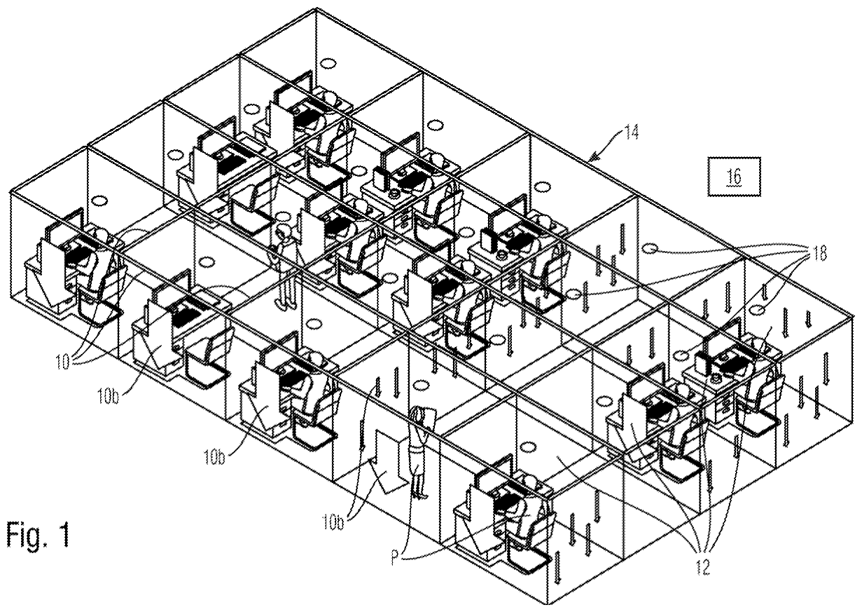

FIG. 1 shows a system for preventing or minimizing the spread of viruses in indoor air according to a first exemplary embodiment of the invention;

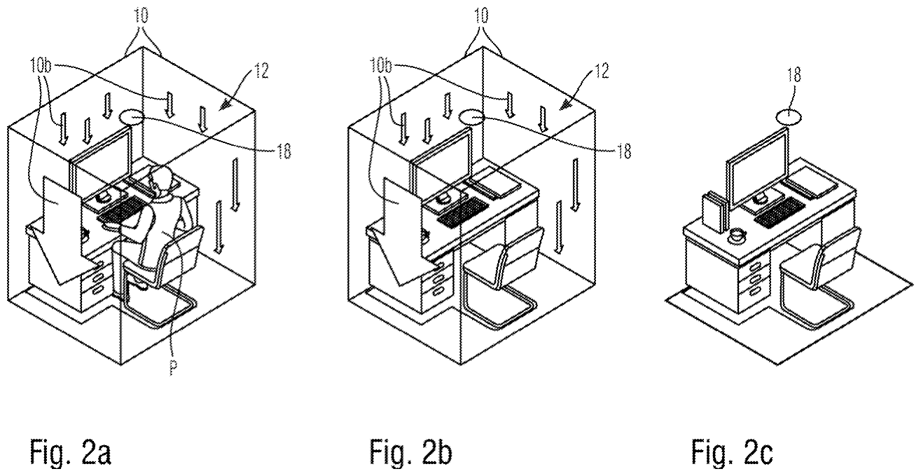

FIGS. 2a through 2c show an individual room segment of the system from FIG. 1 in three different states;

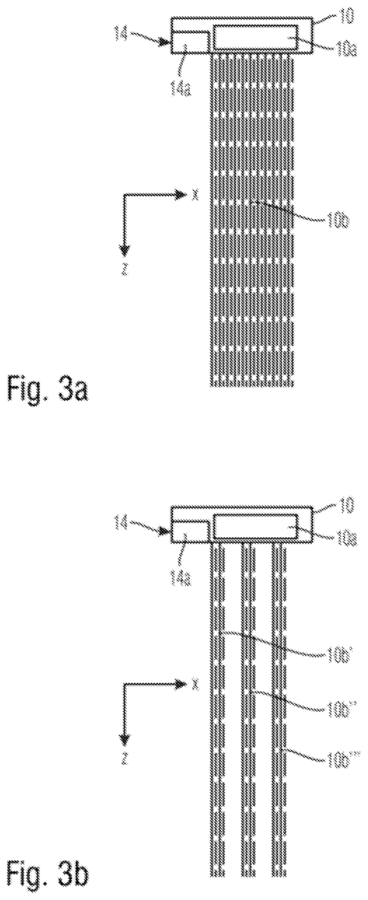

FIGS. 3a and 3b show a schematic sectional view of a radiation source and a wall-like radiation field according to two different exemplary embodiments of the invention;

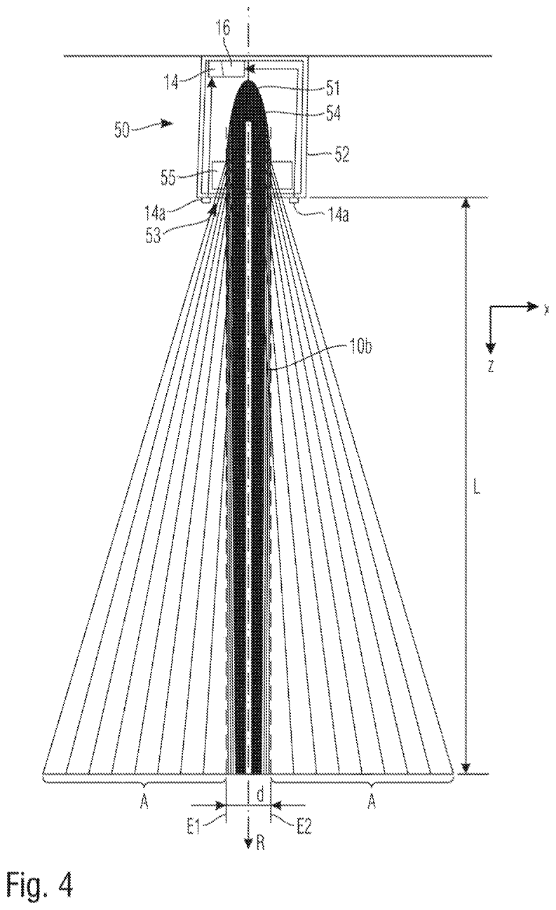

FIG. 4 shows a diagram for explaining the bundling of radiation for generating the wall-like radiation field as a barrier;

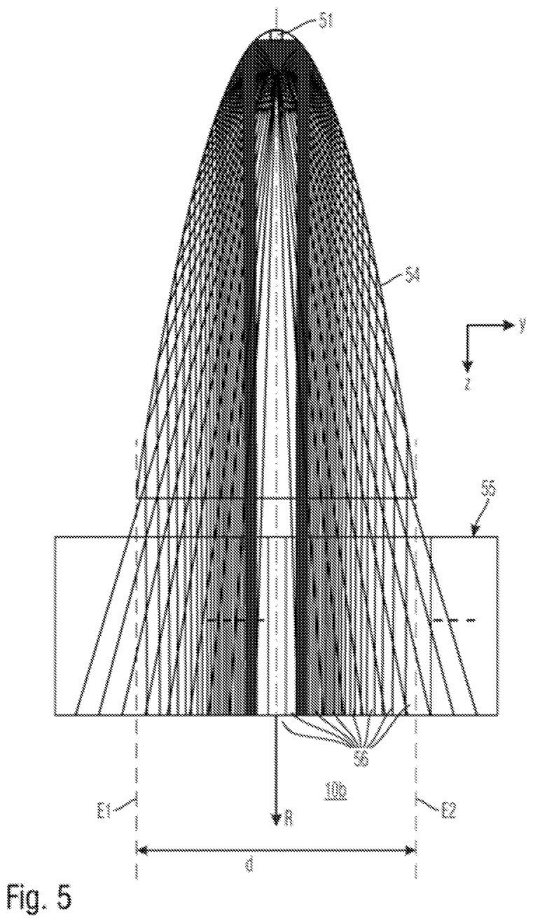

FIG. 5 shows an enlarged illustration of a detail from FIG. 4 for explaining the functioning of the screening device;

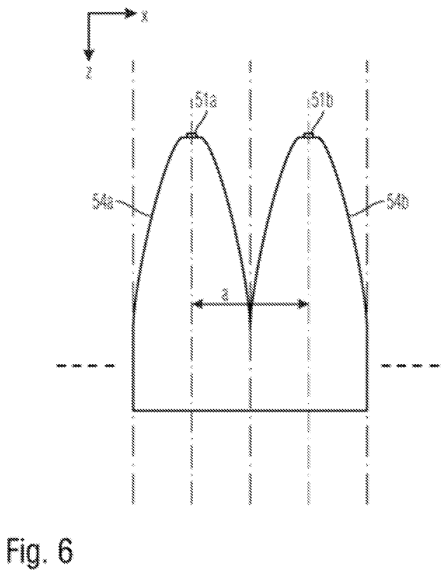

FIG. 6 shows an example of an arrangement of optical elements of neighboring illuminants;

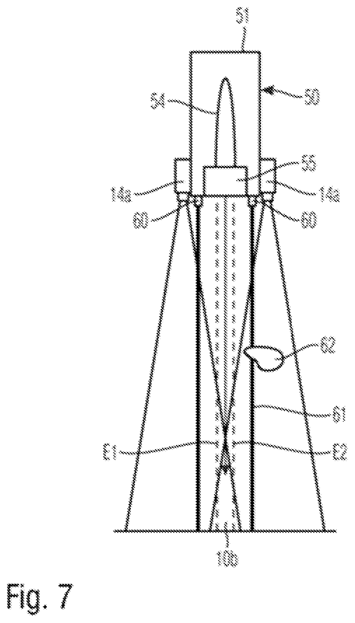

FIG. 7 shows a section through the generated radiation field, with an illustration of a safety zone that is monitored by a sensor system;

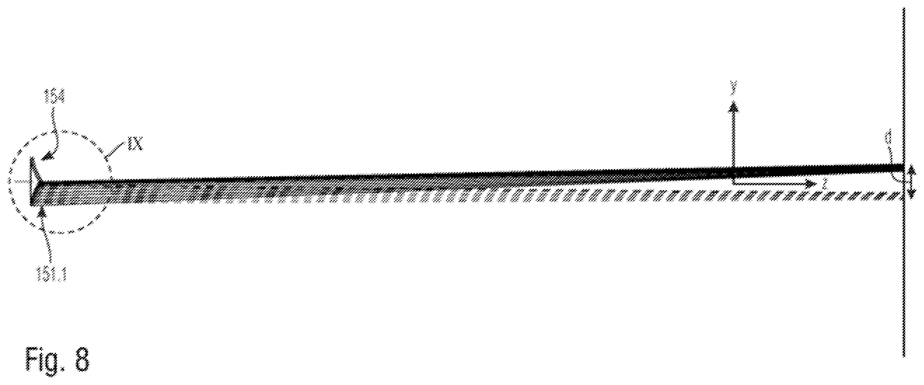

FIG. 8 shows an illustration for explaining an arrangement including multiple illuminant elements for jointly forming a section of the wall-like radiation field by use of a reflector unit;

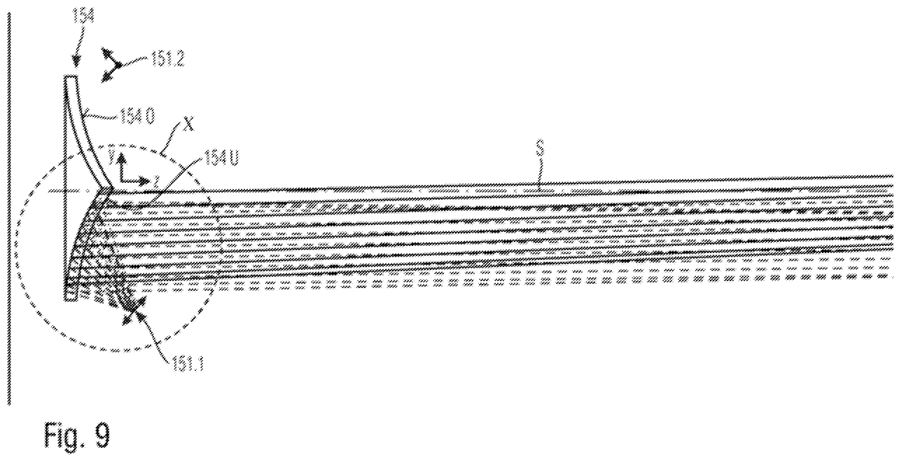

FIG. 9 shows an enlarged illustration in the detail IX from FIG. 8;

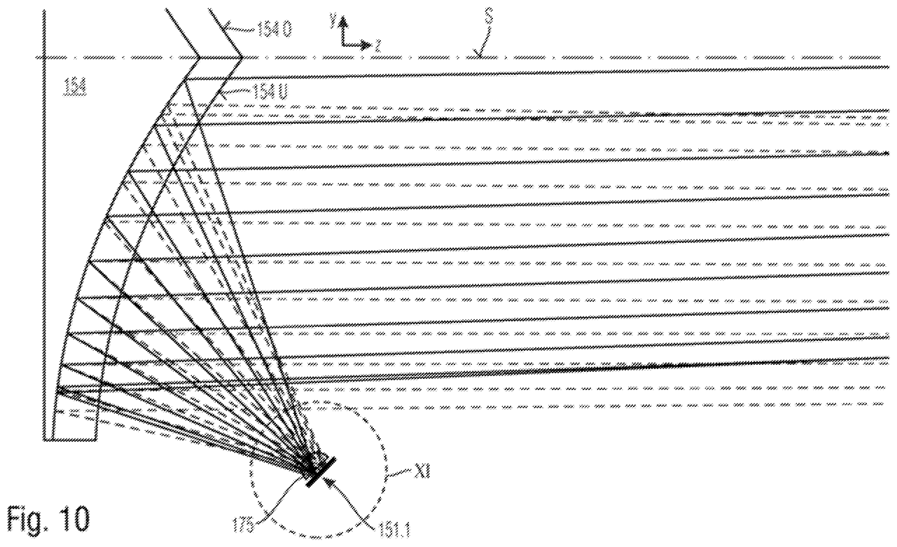

FIG. 10 shows an enlarged illustration in the detail X from FIG. 9;

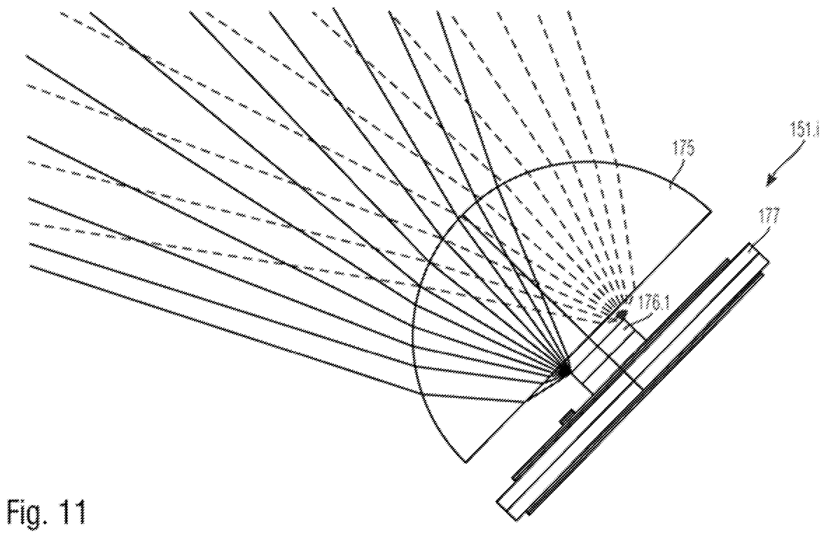

FIG. 11 shows an enlarged illustration in the detail XI from FIG. 10;

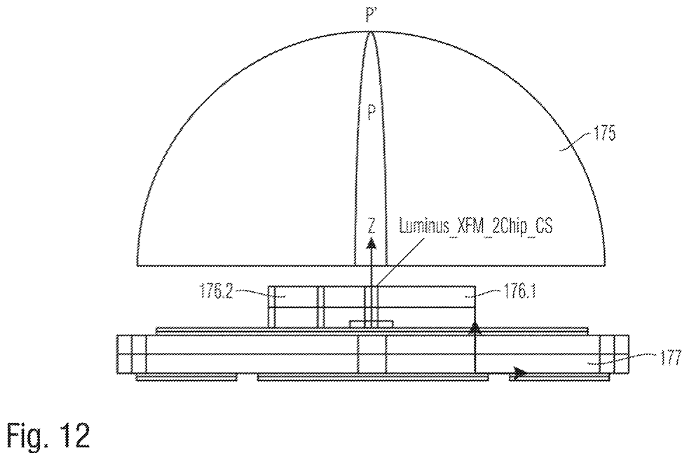

FIG. 12 shows a rotated view of the arrangement of the lens and illuminant element from FIG. 11;

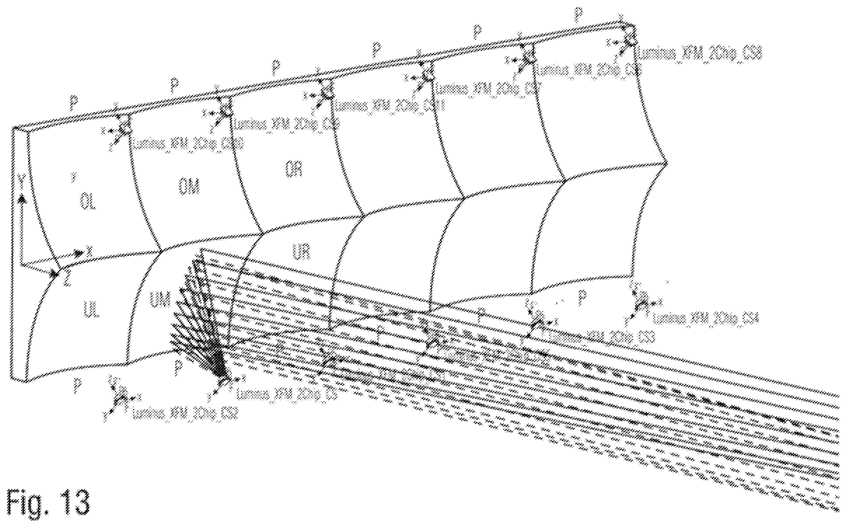

FIG. 13 shows a perspective illustration of a reflector unit;

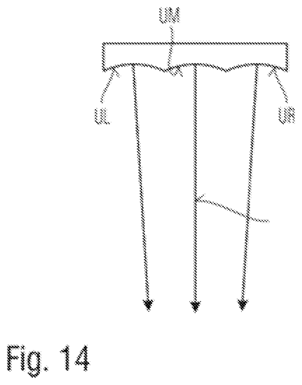

FIG. 14 shows an illustration of reflector surfaces of an assembly;

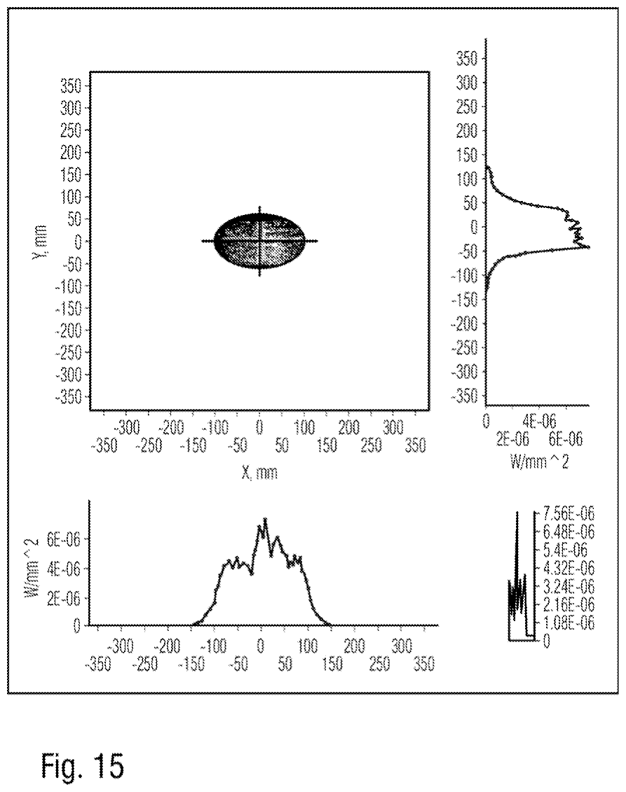

FIG. 15 shows an illustration of an illumination intensity distribution for a first reflector partial surface of the reflector unit;

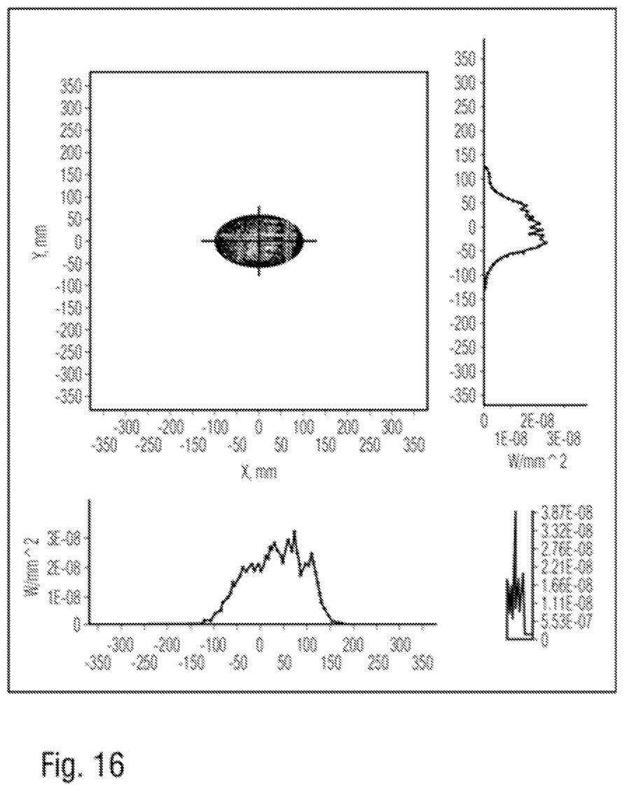

FIG. 16 shows an illustration of an illumination intensity distribution for a second reflector partial surface of the reflector unit;

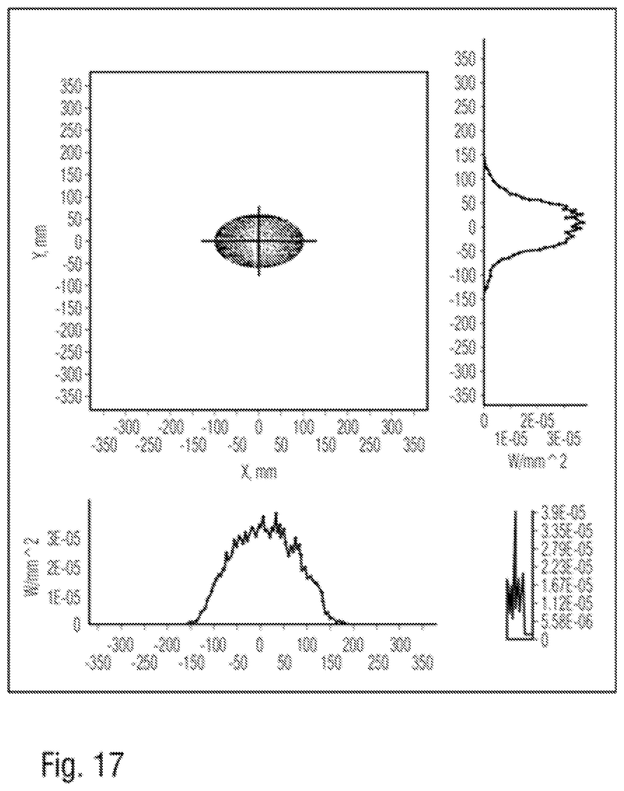

FIG. 17 shows an illustration of an illumination intensity distribution for the entire reflector unit;

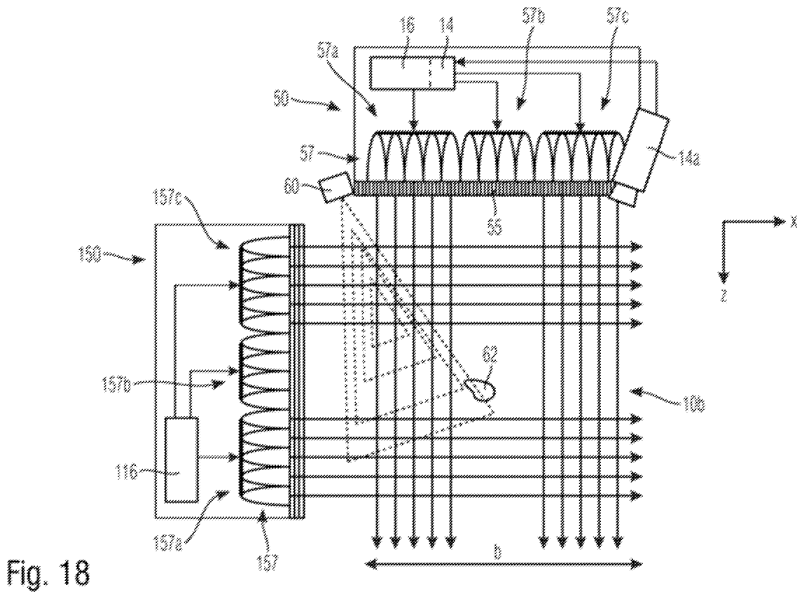

FIG. 18 shows a side view for explaining the selective switching off of subgroups when penetration of an object into the safety zone is detected; and

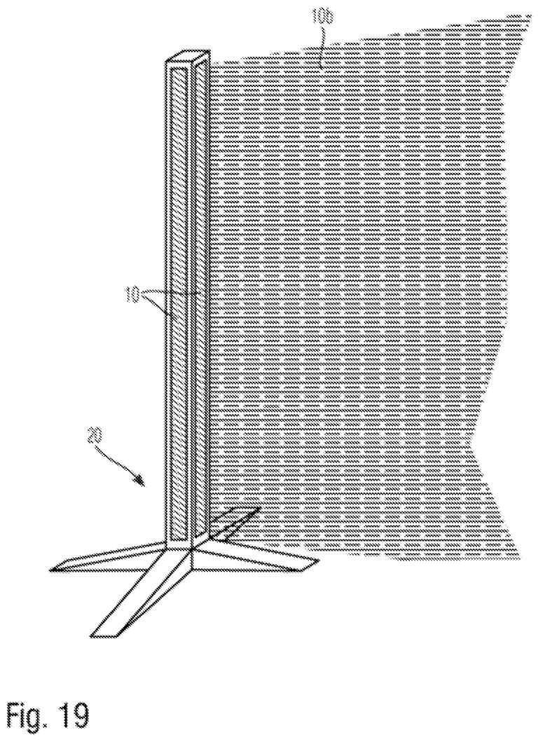

FIG. 19 shows a further embodiment of the invention, including a stand for the radiation sources of a system according to the invention.

Before discussion of one specific embodiment for explaining a lamp according to the invention for achieving, as a result of the invention, the protection of persons in a room from infection with pathogens that are transmittable via air, the system that is set up using the lamp according to the invention is first explained.

FIG. 1 shows a first exemplary embodiment of a system according to the invention, in particular a system for preventing or minimizing the spread of viruses in indoor air in an open-plan office. The open-plan office has a layout that is divided into square room segments, with workstations and corridors situated in rows. Each workstation is equipped with a desk, a chair, and shelves. However, the invention is also applicable to other rooms, for example those with differently sized workstations, or offices with an open-space concept.

A grid-like arrangement of radiation sources 10 is mounted on the ceiling of the room. Each of the radiation sources 10 is a light strip that includes one or more UV-C radiators 10a (FIGS. 3a, 3b), for example mercury vapor discharge lamps, LEDs, or laser diodes, and in each case generates a wall-like radiation field 10b. The use of LEDs or laser diodes is particularly advantageous, since very narrow radiation fields may thus be generated which act as a barrier between the room segments. Thus, pathogens that may be given off by an infected person in one room segment cannot pass through this barrier and into a neighboring room segment. The radiation field 10b may in particular contain short-wave far UV-C radiation having wavelengths in the range of 207-222 nm. Harmful wavelengths may be filtered out via suitable filters. The use of LEDs or laser diodes allows the filters, which are otherwise necessary for protection from the formation of ozone, to be dispensed with. LEDs are available with sufficiently narrow bandwidths, so that a wavelength range may be selected that is completely above the wavelength of 242 nm that is critical for the formation of ozone, and is still short-wave enough for the desired sterilization effect. In this range, the efficiency of the LEDs is also great enough to achieve the necessary irradiation intensity. In particular excimer lamps containing a Kr—Cl gas mixture are suitable for generating the far UV-C radiation. For the sake of simplicity, the wall-like radiation fields 10b are also referred to below as a UV-C wall. The UV-C walls 10b, which are actually invisible, are illustrated in FIGS. 1, 2a, and 2b as vertically downwardly directed white arrows.

For generating a wall-like radiation field 10b, radiation may be bundled or collimated to form parallel beams, optically or by use of slit diaphragms, as explained in greater detail below with reference to FIGS. 4 through 17. Alternatively or additionally, the radiation field 10b may be generated by an arrangement of parallel laser beams having laterally overlapping radiation profiles. Another alternative would be one or more laser beams that are moved back and forth or scanned quickly in a surface, similarly as for barcode scanners, with the scanning speed and the beam diameter being coordinated with one another in such a way that each aerosol that diffuses through the UV-C wall 10b is exposed to a sufficient dose of radiation.

The room segments 12 are each delimited from one another by UV-C walls 10b. In the exemplary embodiment illustrated in FIG. 1, each room segment 12 is bordered by four UV-C walls 10b.

Even though such special cases are not illustrated in FIG. 1, solid walls of the room that are present form boundaries of the room segments 12, so that room segments 12 at the edge or in corners of the room, in addition to the solid walls that are present, need to be delimited only by two or three further UV-C walls 10b. Embodiments of the invention are also conceivable in which a room that is delimited by solid walls, for example a single office, an acoustically shielded meeting area, etc., forms a room segment 12 that can exchange aerosols with other room segments 12 only through a door opening or an entrance. In this case, it is sufficient to shield only the door opening or the entrance in question from the remaining room segments 12 via a UV-C wall 10b.

Structures such as half-height walls, room dividers, or the like may be continued up to the ceiling or expanded via a UV-C wall 10b. In this case, the radiation sources 10 could also be mounted on the top side of the structure in question, and could radiate upwardly toward the ceiling.

In addition, sensors 14a (FIGS. 3a, 3b) of a sensor system 14 for detecting a movement or a presence of one or more persons P in the room are situated in the light strips 10.

A central controller 16 is designed, via suitable software, to switch the one or more radiation sources 10, or at least portions of an individual radiation source 10, on or off as a function of at least the presence of the person P, as described in greater detail below. For this purpose, the controller 16 communicates with the radiation sources 10 via signal lines or wirelessly, for example via WLAN.

The controller 16 evaluates the position data and movement data of the persons P and computes likelihoods for various paths or movements of the persons P. When a person P is sitting quietly at his/her workstation and at a sufficient distance from all UV-C walls 10b, it is unlikely that within the next fraction of a second he/she will pass through one of the UV-C walls 10b. However, if the person is walking quickly through a corridor that is divided into room segments 12 by multiple UV-C walls 10b, the point in time of passing through the next UV-C wall 10b is easily predictable. Because of the health risks, the radiation sources 10 are switched off even for a small likelihood; due to the lower risks, the threshold value for the use of far UV-C radiation may be set to a higher value than for longer-wave types of UV radiation.

If a sufficient likelihood for passing through the UV-C wall 10b has been established based on the movement data detected by the sensor system 14, the controller 16 switches off the radiation source 10 in question, or at least portions thereof. For switching off only a portion, a totality of illuminants that are provided as radiators in the radiation source 10 are divided into groups and optionally subgroups, as described below in the detailed explanation of a lamp as a radiation source 10. In contrast, if an illuminant that extends longitudinally in the radiation source 10 is used, it is only possible to switch off the entire illuminant. Alternatively, switchable screens may be provided, with which certain areas may be shaded.

The persons P may therefore move freely in the room. If the person P passes through a boundary surface between two room segments 12, the controller 16 switches off the UV-C wall 10b that forms this boundary surface, and switches the UV-C wall 10b back on when the person is completely in the second room segment 12.

While one or more persons P are present in the room segment in question 12, the particular radiation sources 10 generally remain active, so that viruses and bacteria in droplets or aerosols are killed upon leaving the room segment 12. Persons P who are present in different room segments 12 are thus shielded from one another by means of the radiation field that forms the barrier. Since the radiation sources 10 remain active while persons P are present in the room segments 12, absorber strips that absorb the incident UV-C light from the radiation sources 10 may be affixed to the floor in order to avoid damage to health due to scattered light.

The radiation source 10 associated with the corresponding UV-C wall 10b is deactivated only when a person P wishes to enter or leave the room segment 12 through a UV-C wall 10b.

As an alternative or in addition to the above-described detection of movements of a person in the room, it is preferably provided that the penetration of a person or an object into a safety zone provided directly adjacent to the radiation field is detected by the sensor system. Such a procedure is explained in greater detail below with reference to FIGS. 7 and 18. A penetration of any given objects into the safety zone is evaluated not only to prevent direct irradiation of a person or a body part of a person, but also to avoid a possible reflection via which persons who are present, even if they are situated at a distance from the radiation field, could likewise be damaged. Monitoring a safety zone that is provided directly adjacent to the UV wall has in particular the advantage that a movement that comes very close to this safety zone does not yet result in switching off at least a portion of the UV wall. One conceivable scenario is the arrangement of the radiation source 10 according to the invention or of the lamp, described in greater detail below, above a table in a restaurant. Typical movements that are made by persons sitting at this table take place in areas that have a sufficient distance from the UV wall. In contrast, if a person reaches over the table, for example to hand something to an oppositely seated person, this is recognized upon penetration into the safety zone, and the appropriate portion or the entire radiation source 10 is switched off. Thus, together with the small thickness of the radiation field, a barrier is built up between persons, without which, distances between persons that are together in a room must typically be increased. The formation according to the invention of barriers between persons, which prevents pathogens from passing from one person to another, thus allows reliable protection from transmission of diseases between persons, without the persons themselves having to adjust their behavior.

The transport of the pathogens takes place via the air. Typical air movement speeds in indoor spaces do not exceed 0.1 m/s. In order to reliably inactivate pathogens, they must absorb a minimum quantity of energy via the irradiation. For the at least 0.6 m/cm² stated above, the retention time of the viruses or bacteria in the radiation field having the preferred thickness d is sufficiently long to achieve the inactivation. In contrast, in the prior art significantly larger volumes, i.e., also thicknesses of the radiation field, are necessary, since for the lower irradiation intensities typically achieved there, longer retention times are required for killing the viruses or bacteria.

Further radiation sources 18 having a virus-deactivating or disinfecting effect are centrally mounted on the ceiling in the room segments 12.

The controller 16 is designed to activate the further radiation sources 18 for a predefined time interval if no person is present in the room segment 12. These radiation sources 18 are also switched off when a person P enters the room segment 12 in question. In order to indicate to the person P whether the disinfection of the room segment in question 12 is concluded, a light-emitting diode or a “traffic light” system may be provided. Further embodiments of the invention are conceivable in which the sensor system 14 includes sensors that are integrated into the radiation sources 18. The radiation sources 18 may be integrated into ceiling panels, lamps, or ventilation louvers, or integrated into a housing with other devices, for example smoke detectors.

FIGS. 2a through 2c show an individual room segment 12 of the system from FIG. 1 in three different states.

In the work state illustrated in FIG. 2a, a person P is working in the room segment 12 that is delimited by four UV-C walls 10b. All four UV-C walls 10b are switched on, so that pathogens contained in aerosols are inactivated upon passing through the boundary surfaces between adjoining room segments 12.

In the disinfection state illustrated in FIG. 2b, a person P has worked in the room segment 12 and left it. When the person is leaving the room segment 12, one of the four UV-C walls 10b is switched off (not illustrated) due to the detected movement of the person P. All four UV-C walls 10b are switched on so that no active pathogens can escape. In addition, the centrally mounted radiation source 18 on the ceiling is activated for a predefined time period to also kill the pathogens on the surfaces of the workstation and suspended within the room segment 12.

In the rest state illustrated in FIG. 2c, the disinfection is completed and no person P is present in the room segment 12. To save energy, all four UV-C walls 10b and also the centrally mounted radiation source 10 on the ceiling are switched off.

FIG. 3a shows a schematic sectional view of a radiation source 10 and a wall-like radiation field 10b according to the first exemplary embodiment of the invention. The radiation field 10b, within the scope of the optical options, has a constant thickness of approximately 1 cm.

As described above, the controller 16 implements a method for preventing or minimizing the spread of viruses in indoor air, using one or more radiation sources 10 in a room. The method includes detecting a movement or a presence of one or more persons P in the room, and automatically switching one or more radiation sources 10 on or off as a function of at least the presence of the person P.

According to the method, the radiation source 10 in question is switched off when the movement data detected by the sensor system 14 indicate that it is likely that one of the persons P would like to pass through the radiation field 10b in question, or that a person or an object has penetrated into the safety zone.

FIG. 3b shows a further exemplary embodiment of the invention. To avoid repetitions, the following description of these further exemplary embodiments is limited essentially to differences from the first exemplary embodiment of the invention. On account of the unchanged features, one skilled in the art will refer to the description for the first exemplary embodiment. For identical or similarly functioning features of the further exemplary embodiments, the same reference numerals are used in order to emphasize the similarities.

In the exemplary embodiment illustrated in FIG. 3b, the radiation sources 10 are each designed to generate multiple parallel radiation fields 10b′ through 10b′″ which may have a thickness, for example, of less than 50 mm, preferably less than 40 mm, more preferably 25 mm or 1 mm, and a spacing of 1 mm, for example. Greater distances between the parallel radiation fields 10b′ through 10b′″ are possible, although this increases the space requirements. In addition, a different number of radiation fields 10b′ through 10b′″ is conceivable.

For forming the above-described system, lamps 50 that correspond to the embodiments illustrated in FIG. 4 are preferably used as a radiation source 10. It should be noted that the illustrations are strictly schematic, and no assertion is made concerning a correct rendering of proportions. Rather, where deemed appropriate, the proportions are adapted to make the invention easily understandable.

The lamp 50 illustrated in FIG. 4 includes a plurality of illuminants 51, only one of the illuminants 51 being discernible in FIG. 4 due to the sectional illustration. The lamp 50 also includes a housing 52 that is impermeable to UV-C radiation. The housing 52 has an exit opening 53 through which UV-C radiation generated by the illuminant 51 can exit from the lamp housing 52. In the illustrated exemplary embodiment, the lamp 50 is provided for mounting on a ceiling of a room. Of course, mounting on a wall of a room may also take place. The function described below is independent of the orientation of the lamp 50.

The illuminant 51 emits germicidal UV-C radiation, which is collimated by a reflector 54. The reflector 54 is one example of an optical element via which radiation that is emitted by the illuminant 51 may be collimated. Other optical elements, for example appropriately designed lenses, are likewise conceivable. The selection and development of the optical element used for collimating the emitted radiation may take place based, for example, on economic or manufacturing aspects or the efficiency.

The radiation that is reflected at the inner side of the rotationally symmetrical reflector 54 is referred to as collimated radiation. This collimated portion of the radiation that is emitted by the illuminant 51 leaves the exit opening 53; due to the collimation, the collimated radiation leaves the exit opening 53 within an imaginary cylinder, having the diameter d, in the direction of the z axis. The geometry of the reflector 54 is selected in such a way that for typical room heights or room dimensions, which may be estimated using a maximum length L equal to 5 m, the diameter d of the collimated radiation is always less than 8 cm, preferably less than 5 cm. It should be noted that these data are strictly preferred values. To allow such a small extension in the transverse direction with respect to the radiation direction, it is preferred to use LEDs as the illuminant 51. The irradiation intensity achieved within the diameter d is greater than 0.6 mW/cm², which ensures that pathogens penetrating into the radiation field of the barrier are reliably killed. Unlike systems known from the prior art which irradiate a large air volume in each case, the pathogens may already be inactivated over the small distance due to the thickness of the radiation field, which corresponds to the diameter d of the collimated radiation.

The longitudinal axis of the lamp 50 is perpendicular to the plane of the drawing. The arrangement of the illuminant 51 and of the reflector 54, illustrated in cross section, is repeated along the longitudinal axis of the lamp 50, with the plurality of illuminants 51 and their associated reflectors 54 situated in the lamp 50 being arranged along a line, preferably a straight line. In the embodiment illustrated in FIG. 4, the illuminants 51 and their associated reflectors 54 situated in the lamp 50 thus jointly form a single group, the radiation directions R of all individual illuminants 51 and their associated reflectors 54 being parallel to one another and lying in a plane. Alternatively, the radiation directions may also lie in a curved surface, although a plane is preferred. Therefore, without limiting generality, representative reference to a plane is made below.

As will be explained in greater detail below, the neighboring reflectors 54 are arranged along this line in such a way that the radiation collimated in each case within the diameter by the neighboring reflectors 54 is directly adjacent and overlaps the radiation in the areas A, and the totality of the collimated radiation of the illuminants 51 thus generates the wall-like radiation field 10b as a barrier for viruses. The maximum extension of this wall-like radiation field 10b in a direction perpendicular to the longitudinal extension of the lamp 50 and to the radiation direction, i.e., the extension in the direction of the y axis, is bounded by two imaginary planes E1 and E2. The distance between these two planes E1 and E2 thus corresponds to the diameter d of the imaginary cylinder.

The illuminants 51 and the reflectors 54 are coordinated with one another in such a way that the intensity of the collimated radiation is sufficient to kill pathogens, and as stated above, in particular is greater than 0.6 mW/cm². In contrast, outside the UV wall 10b thus formed, radiation is present only with a noncritical intensity. This radiation results from the uncollimated portion of the radiation that is emitted by the illuminant 51, i.e., the portion that exits from the reflector 54 without reflection. In FIG. 4, this radiation component is shown by individual beams outside the area between the planes E1 and E2. The radiation intensity in the areas A is so low that health damage to persons is ruled out.

For enhancing safety, a screening device 55 is preferably situated in the area of the exit opening 53 of the lamp 50. The screening device 55 itself may form the exit opening 53, or it may be situated inside or also outside the housing 52 of the lamp 50. The operating principle of the screening device 55 is explained in greater detail below with reference to FIG. 5. The screening device 55 ensures that the uncollimated portion of the radiation emitted by the illuminant 51 is shaded, i.e., hindered at the exit from the opening 53. As illustrated in FIG. 4, this portion radiated directly by the illuminant 51 outside the UV wall that is bounded by the planes E1 and E2 would illuminate the areas A. Thus, if a critical intensity of the UV-C radiation present there occurs, a person could not be present in these areas without a safety risk. Regardless of the exact positioning of the screening device 55, the screening device 55 is dimensioned and positioned in such a way that all radiation leaving the housing 52 of the lamp 50 must pass through the channels of the screening device 55.

FIG. 4 also illustrates that sensors 14a, which are part of a sensor system whose information processing may be integrated into the controller 16, are situated at the lamp 50. In the illustrated exemplary embodiment, the controller 16 is integrated into the lamp 50. However, at least signals of the sensors 14a or even a result of an evaluation are/is transmitted to the controller 16, so that the controller may switch the illuminants 51 on or off based on the evaluated signals.

FIG. 5 shows an enlarged illustration of the illuminant 51 and the reflector 54 together with the screening device 55. Schematically shown are channels 56 of the screening device 55 which extend in parallel to the radiation direction R and thus allow the collimated radiation to pass through, while radiation components extending at an angle to the radiation direction R strike inner walls of the channels 56. To ensure that no risk arises, even from a beam that is possibly reflected at an inner wall, the inner walls of the channels 56 are coated with a material that absorbs UV-C radiation, or the screening device 55 is made of such a material.

The screening device 55 may be provided either individually for each reflector 54, and for example cover the opening of the reflector 54, or may be provided as a shared screening device for the totality of the reflectors 54.

In addition, it is to be noted that for the detailed explanation of the lamp 50, it is assumed that a plurality of individual illuminants 51 jointly emit the radiation that ultimately forms the UV wall. However, an illuminant that extends in the longitudinal direction may also be used for generating the radiation.

FIG. 6 shows, in a greatly simplified manner, a section through the reflective surface of neighboring reflectors 54 in the form of a first reflector 54a and a second reflector 54b. The two reflectors 54a and 54b are spaced apart in the lamp 50 by a distance a that is smaller than the diameter d of the imaginary cylinder or the distance d between the imaginary planes E1 and E2 as boundaries of the wall-like radiation field 10b.

In the illustrated exemplary embodiment, it is assumed that all reflectors 54 that are provided in a lamp 50 have identical geometries. The collimated radiation that is thus emitted in each case by an illuminant 51 with the aid of its associated reflector 54 is thus the same with regard to its radiation geometry. In principle, it is also conceivable to use different geometries for neighboring reflectors 54. The distance between the particular axes of symmetry when rotationally symmetrical reflectors are used is then to be adapted in each case so that the imaginary cylinders that envelop the collimated radiation intersect. In order for an overlap of the collimated radiation to reach neighboring reflectors, the neighboring reflectors may also be arranged in such a way that their radiation directions enclose a small angle relative to one another. In particular the first, third, fifth, etc., reflectors are situated so that their radiation directions are in parallel to one another, but the radiation directions of the second, fourth, sixth, etc., reflectors enclose an angle, with their radiation directions once again being in parallel to one another.

As indicated above, for operating the lamp 50 according to the invention or the overall system, the emitted UV radiation must be reliably prevented from striking persons who could thus be harmed. In addition to the prediction of movements of persons, or detections of the location at which persons are present, explained above in conjunction with the system, the direct penetration into a safety zone, defined as adjacent to the radiation field, i.e., neighboring the planes E1 and E2, may be detected. FIG. 7 shows a greatly simplified illustration of a sensor system via which the penetration into such a safety zone may be detected.

In the illustrated exemplary embodiment, reflections that result when radiation that is emitted by a so-called line laser 60 strikes a surface are detected by means of sensors 14a. In the illustrated exemplary embodiment it is assumed that persons may be present on both sides of the UV wall 10b, as is typical in a restaurant. Therefore, a line laser 60 and an associated camera as a sensor 14a for detecting the reflections of the laser beam are provided on both sides of the UV wall 10b. To the left of the UV wall 10b it is apparent that the emitted laser radiation of the line laser 60 situated on the left falls, for example, on the floor or some other essentially unchangeable device objects. This reflection is detected by the sensor 14a.

In contrast, on the right side of the UV wall 10b it is shown that an object 62, which may be, for example, a finger of a person or an object that is moved by the person, approaches the UV wall 10b and thus comes into an area, in that it reflects a portion of the laser light that is radiated by the line laser 60. Up to the time of penetration into the plane of the laser light that is emitted by the line laser 60, here as well the light has been reflected solely from the floor. In contrast, the reflection changes immediately upon penetration by the object 62, which is detected by the sensor 14a. Based on the change, the penetration of an object into the safety zone may be deduced. The safety zone is the space from the UV wall 10b or the adjoining plane E2 up to and including the radiation that is emitted parallel to the plane E2 by the line laser 60 situated on the side of the plane E2.

A safety zone is likewise formed on the side of the other plane E1. The formation of a second safety zone may be dispensed with if the lamp is mounted close to a wall and parallel thereto, so that penetration into the area of the UV wall 10b is impossible from this side.

When the lamp 50 generates multiple radiation fields 10b′ through 10b′″ situated in parallel, the safety zones are to be provided only adjoining the respective outermost radiation field. The increasingly larger distances on the end-face side due to the multiple radiation fields 10b′ through 10b′″ are then to be safeguarded using separate protective measures. These may correspond to the above-described safety devices situated in parallel to the radiation fields. If the extension of the lamp extends between two walls or other structural objects that shield UV light, safeguarding the end-face sides may also be dispensed with.

The above statements in each case assume that the light wall may be built using an individual illuminant and an assigned reflector, with multiple such units in a consecutive series. The shown arrangement positions the illuminant in the center of the reflector. However, such a design is problematic with regard to the achievable irradiation intensities. In particular, in this simple arrangement the extension of the illuminant, i.e., at least the radiating surface of an LED, for example, also ensures that the delimitation between the illuminated surface within the thickness d and its neighboring area A is very indistinct. However, it is desirable for the area that is active for killing the pathogens to be delimited from its surroundings as sharply as possible. Therefore, an arrangement as described below is preferred, in which multiple illuminant elements and their associated optical devices (reflector partial surfaces of a reflector unit) are combined to form a UV radiator unit. The structure of the overall wall-like radiation field is obtained by situating multiple of these UV radiator units in succession. In other respects, the above statements also apply to a system that generates the wall-like radiation field using the arrangement described below.

Firstly, FIG. 8 illustrates a cross section of a reflector unit 154 of the UV radiator unit, the beam path generated using an individual partial surface of the reflector unit 154 and its associated illuminant element 151.1 being schematically illustrated. The illuminant element 151.1 used in the illustrated exemplary embodiment is an LED with two LED chips, which are situated in succession in the direction of the x axis. This arrangement is explained in greater detail below with reference to FIGS. 11 and 12. However, the precise design of the radiating surfaces is not limiting for the invention. Thus, it is also conceivable in particular to use only one chip per LED, depending on the further development of LED technology, if the radiation power thus generated is sufficient, or to use multiple chips situated in some other way. The reflector unit 154 has a plurality of reflector surfaces 154 U, 154 O, which are explained in greater detail below with reference to FIGS. 9, 10, and in particular also FIG. 13.

It is clearly apparent in FIG. 8 that the reflector unit 154 has a symmetrical design, with its plane of symmetry situated in the x-z plane. In FIG. 9 the plane of symmetry is denoted by reference symbol S and is illustrated as a dash-dotted line. The beam patterns shown in FIG. 8 emanate from the lateral boundaries of the LED chips that generate the UV radiation. With the aid of a hemispheric lens, explained in greater detail below with reference to FIG. 11, the emitted UV radiation is imaged onto an illuminated surface, where it has an extension d in the y direction which is not greater than 120 mm, for example. This extension d is a depiction of the width of the radiating LED chip(s) in the y-z plane. It is apparent in FIG. 8 that, although only one-half the reflector unit 154 is irradiated by the illuminant element 151.1, the irradiated area is situated on a surface that is perpendicular to the plane of symmetry S and contains a focal point of the reflector surfaces 154 U, 154 O that is symmetrical with respect to the z axis. This correspondingly applies to the nonirradiated partial surface of the reflector unit 154 in FIG. 8. It is thus ensured that the partial surfaces of the reflector unit 154, irradiated on both sides of the plane of symmetry S, reflect the reflected UV light in the y direction onto the same area having the thickness d. This is achieved by slightly tilting the reflector surfaces, so that the two focal points of the reflector surfaces 144 [sic; 154]U, 154 O coincide.

FIG. 9 shows an enlarged illustration of the detail IX from FIG. 8. It is apparent that the light emitted by the illuminant element 151.1 is reflected at a first reflector surface 154 U. The depicted dashed or dotted lines represent the beam patterns of the right or left edge (in the y direction) of the UV light-emitting chips of the illuminant element 151.1. It is apparent in the upper half of the illustration that a second reflector surface 154 O, which is symmetrical with respect to the y-z plane (plane of symmetry S), is provided. To elucidate the position of the illuminant elements 151.1 and 151.2, a further such illuminant element is schematically indicated at the location designated by reference numeral 151.2. The arrangement and orientation of the illuminant elements 151.1 and 151.2 are likewise symmetrical with respect to the x-z plane.

It is also apparent in FIG. 9 that the illuminant elements 151.1 and 151.2 are situated outside the area in which the incident radiation is reflected by the two reflector surfaces 154 U and 154 O. In this way, shielding (shading) of the radiation reflected by the reflector unit 154 may be avoided, and an undesirable reduction of the irradiation intensity at the illuminated surface or in the generated radiation field in general is prevented. However, it is to be noted that a smaller angle between the z axis and the center axis of the radiation emitted by the illuminant element 151.1 on the one hand may be advantageous with regard to the further radiation profile, and on the other hand allows a smaller overall width.

FIG. 10 shows yet another enlarged illustration in the area X from FIG. 9. In addition to the illuminant element 151.1, the hemispheric lens 175 is also apparent here. The use of a hemispheric lens 175 has in particular practical advantages, since such lens geometries are readily available at an economical price. For the same reason, the reflector surfaces 154 U and 154 O are partial surfaces of ellipsoids. One focal point of the ellipsoid is situated in the area of the LED chip whose radiation is to be reflected (from a geometric standpoint, thus within the radiating volume, including its boundary surfaces), and the other focal point is situated at the intersection point of the z axis with the illuminated surface. The “illuminated surface” may be a reference surface which, as a function of the mounting and the actual distance from the illuminated surface during operation, coincides with same. For room heights up to 5 m, this reference surface may be provided at a distance of 2.50 m to 5 m. Since this condition applies for all reflector surfaces, the two reflector surfaces 154 U, 154 [O] situated symmetrically with respect to the x-z plane illuminate the same area having a width d. Although the reflector surfaces 154 U, 154 O are shifted relative to the z axis, as the result of a slight inclination of the optical axis of the individual reflector surfaces 154 U, 154 O with respect to the z axis, the same area of a surface that is perpendicular with respect to the axis of symmetry S and that extends through the focal point of the reflector surfaces 144 [sic; 154] U, 154 O is illuminated in they direction over both reflector surfaces 154 U, 154 O.

FIG. 11 shows an enlarged illustration of the detail XI from FIG. 10. It is apparent that an enlarged image of the LED chip 176.1 is generated by the hemispheric lens 175. It is also apparent that the beams shown for explaining the principle in FIGS. 8 through 10 emanate from the edges, i.e., the lateral ends (with respect to the y-z plane) of the LED chip 176.1. It should be noted that radiation is emitted not only from the surface of the LED chip 176.1 facing the hemispheric lens 175, but also from its lateral boundary surfaces. The LED chip 176.1 is situated on a substrate 177. This design is identical for all illuminant elements 151.i used.

FIG. 12 shows an illustration of the hemispheric lens 175 and of the illuminant element 151.i rotated by 90°. In this rotated illustration, it is apparent that the illuminant element 151.i has a second LED chip 176.2 situated adjacent to the first LED chip 176.1. The two LED chips 176.1 and 176.2 are arranged in such a way that their longitudinal extension is parallel to the x axis. As explained above, the hemispheric lens 175 generates an enlarged image of the LED chip surface area that results from the two LED chips 176.1 and 176.2. The surface of the individual LED chips 176.1 or 176.2 facing the hemispheric lens 175 is square, and has an edge length of 1 mm. This results in a rectangular total chip surface area of 2 mm times 1 mm. The orientation of the adjacently situated LED chips 176.1 and 176.2 is such that the extension d, as explained for FIG. 8, [has] an image that corresponds to the width of the LED chips 176.1 and 176.2. In contrast, the image of the longitudinal extension (2 mm) of the overall surface of the LED chips 176.1 and 176.2 extends along the x axis, as explained in greater detail below.

The above statements in each case relate to a reflector surface 154 U, with a plurality of reflector surfaces and their associated units, made up at least of the illuminant element 151.i and the hemispheric lens 175 situated in front of same, cooperating to form a UV radiator unit. FIG. 13 shows a perspective illustration of two such UV radiator units, each including six reflector surfaces and having a symmetrical design, which are situated in the longitudinal direction of the lamp, i.e., parallel to the x axis in the drawings. The six reflector surfaces of the left UV radiator unit are denoted by reference symbols UL, UM, UR and OL, OM, and OR, the reflector surfaces designated by U and their associated units belonging to a first group, and the reflector surfaces designated by 0 and their associated units belonging to a second group. In the illustrated embodiment, the first group and the second group are symmetrical with respect to a center plane of the lamp and are situated directly adjoining one another. The center plane coincides with the plane of symmetry S of the reflector. The above-described mutually tilted orientation of the reflector surfaces results in the advantageous superimposition of the respective radiation portions that are reflected by the individual groups. The two reflector surfaces, situated opposite from one another, have a shared focal point. However, with the increasing available power of LEDs, it is also conceivable to provide a single-row arrangement, i.e., only one of the two groups. If two symmetrically arranged groups are provided as in the illustrated exemplary embodiment, a distance may also be provided between these two groups.

The beam path in FIG. 13 is illustrated only for the reflector partial surface UM in order to not detract from the clarity of the reflector units illustrated in perspective. In one preferred embodiment, the grid dimension for the reflector surfaces in the x direction is 70 mm. The middle reflector surfaces OM, UM are thus situated at x=0. The neighboring reflector surfaces UL and OL are at −70 mm, or UR and OR are at +70 mm. The reflector unit 154 thus has an overall length of 210 mm in the x direction.

Each reflector surface extends 60 mm in the Y direction, so that the overall width of the reflector unit in the Y direction is 120 mm. These dimensions (120 mm times 210 mm) correspond to the illuminated surface at a distance of 2500 mm from the reflector unit 154 (reference surface). This distance is measured starting from the rear-side, shared mounting plane of the overall reflector unit. Since the surface of the reflector unit 154 and the irradiated surface are the same size, the extension of the wall-like radiation field may be enlarged by situating multiple UV radiator units in a consecutive series, without at the same time increasing their thickness.

FIG. 14 shows a longitudinal section of three reflector surfaces UL, UM, and UR that form an assembly. It is apparent that the two outer radiation directions of the reflector surfaces UL, UR are oriented toward the center, with all three radiation directions lying in a plane. The radiation direction of the middle reflector surface UM is then referred to as the radiation direction R of an assembly. The illustrated examples in each case combine three LEDs to form an assembly. However, this is not limiting. Alternatively, two LEDs together with their associated reflector surfaces, or four or more LEDs together with their associated reflector surfaces, may in each case be combined to form an assembly. In this case, a symmetry line for which the reflector surfaces are situated symmetrically on both sides is referred to as the radiation direction. Alternatively, the illuminants for the two outer reflector surfaces UL and UR, explained in greater detail below, may also be situated at a slightly greater distance from the illuminant of the middle reflector surface UM than the grid dimension in order to achieve the same effect.

FIG. 15 shows the pattern of the illumination intensity in the x and y directions for a single illuminant element 151.1, which is situated in relation to the reflector surface UM. It is apparent that the rectangle, situated symmetrically with respect to the origin of the x-y plane, is irradiated by this illuminant element 151.1. However, light that is [emitted] by a further illuminant element that is associated with the reflector surface UR also illuminates the same rectangular surface. The reason is the arrangement of the illuminant unit [sic; element] 151 that is slightly shifted with respect to the symmetry of the reflector surface in the direction of the x axis. While the illuminant element for the middle reflector partial surface UM is situated centrally, in the x direction, above the reflector partial surfaces, the two illuminant elements situated on the outside are positioned with a slight offset, so that the distance from the illuminant units [sic; elements] of the middle reflector surfaces is greater than the grid dimension of the reflector surfaces. This results in centering of the reflected UV radiation, as illustrated for the reflector surface UR in FIG. 17.

Alternatively, as described above, an inclination of the reflector surfaces or illuminant elements could also be provided. However, on the one hand this results in more complex manufacture of the reflector unit 154, and on the other hand, the illuminant elements then can no longer be situated in a shared plane.

A consideration of the illumination intensities that result when all six reflector surfaces reflect light from six associated illuminant elements 151.i results in the distribution of the illumination intensity as illustrated in FIG. 16.

It should be noted that the above statements assume that two LED chips jointly form an illuminant element. However, it is also conceivable for more than two LED chips to form an illuminant element if this plurality, for example three, are likewise arranged in a row.

In such a case, the number of reflector surfaces could even be reduced, since each reflector surface in this case would be illuminated by the light from three LED chips. In consideration of the losses that occur at the illuminated surface, it is crucial that a sufficiently high irradiation intensity is achieved. For a given radiation power of the LED chips, this results in the number of chips required for irradiating a certain surface. It should be noted that the irradiation intensity that occurs at the illuminated surface is merely a measure for describing the power density in the wall-like radiation field. Ultimately, the area between the lamp and the irradiated surface that is illuminated by light is crucial for killing pathogens.

The functioning of the safety devices discussed above is now explained with reference to the illustration in FIG. 18. The arrangement illustrated in FIG. 18 shows the lamp 50, already explained with reference to FIG. 7, together with the sensor 14a and the line laser 60. The laser light emitted by the line laser 60 is schematically illustrated by the dashed-line triangle. The plane in which the laser light is emitted is parallel to and situated at a distance from the collimated radiation that may be emitted by the totality of illuminants 51 of the lamp 50. The reflected portion of the radiated laser light of the line laser 60 is detected by the sensor 14a and supplied to an evaluation. As explained above, in the evaluation in particular the change in the reflection of the laser light is detected, so that a penetration of an object into the area illuminated by the line laser 60 may be detected by the sensor system or its information processing device 14. The sensor system 14 may in particular include a processor or other devices for processing the information transmitted by the sensor 14a. This device for data processing may be implemented jointly with the controller 16. In the illustrated exemplary embodiment, the controller 16, including the information processing portion of the sensor system 14, is integrated into the lamp 50.

In the lamp 50 illustrated as an example, a total of fourteen illuminants 51 are arranged along a straight line, and an optical element in the form of a reflector 54 (illustrated here without a reference numeral) is associated with each of these illuminants 51, which for the sake of better clarity are not separately illustrated in FIG. 8 [sic; 18]. The emitted UV light is representatively indicated by the radiation directions depicted as arrows. The radiation directions of the illuminants and their associated optical elements are oriented in parallel to one another, as is directly apparent from the drawing. In addition, all radiation directions of the illuminants of the lamp 50 are situated in a plane. Thus, all illuminants of the lamp 50 jointly form a group of illuminants.

As an alternative to a lamp 50 as illustrated, which includes only a single group of illuminants, multiple groups of illuminants may also be provided. Within a group, the illuminants and their associated reflectors are then likewise once again arranged in such a way that their radiation directions are parallel to one another and are situated in a plane, or as stated as an alternative above, lie in a surface. The planes (or surfaces) of various groups may be situated in parallel and spaced apart from one another, or they may enclose an angle.

For the group of illuminants and their associated optical elements of the lamp 50, it is shown that the group is divided into three subgroups 57a, 57b, and 57c. Each of these subgroups 57a, 57b, and 57c contains a plurality of illuminants and their associated optical elements. The subgroups 57a, 57b, and 57c may be individually controlled, i.e., switched on and off, by the controller 60.

If an object 62, upon penetration into the plane illuminated by the line laser 60, is now detected based on the signals recorded by the sensor 14a, the position of the object 62 is determined from the signals transmitted from the sensor 14a to the controller 16 or to the information processing device of the sensor system 14 integrated therein.

It should be noted that only one line laser 60 and one sensor 14a are shown in FIG. 18; however, it is particularly preferred to provide a plurality of such combinations of line lasers and sensors 14a, whose detection directions enclose an angle that is not equal to 0° or 180°. It is possible to determine the position of the object 62 in two dimensions by using such a combination of arrangements. Furthermore, when two such arrangements are used, it is also possible to separately detect a further object that is possibly situated in the shadow of the shown object 62.

In contrast, when only one arrangement is used, the position of the object 62 may be determined at least in one direction (x axis). The detected position is evaluated in the controller 16, and the subgroup 57a, 57b, or 57c whose emitted, collimated radiation would strike the object 62 is switched off. In the illustrated exemplary embodiment, this is the middle group 57b. It should be noted that the term “position” is understood to mean not only a midpoint of a detected object 62, but also its extension. Thus, if a detected object 62 is not completely in the area of the light that is radiated by a subgroup 57a, 57b, or 57c, not just one subgroup is switched off, due to the position detection, including the extension of the object 62.

On the other hand, if the position coordinates are known for two directions (x axis, y axis), a second lamp 150 whose design is basically comparable to that of the lamp 50, and whose radiation directions with the radiation directions of the lamp 50 enclose an angle not equal to 0° or 180°, may be used. The radiation directions of the lamps 50 and 150 are preferably perpendicular to one another. The radiation directions of both lamps 50 and 150 are preferably situated in the same plane, so that the sensor system 14, including the line laser 60 and the sensors 14a, may be jointly used. If the position of the object 62 is determined in two dimensions with the aid of the sensor system 14, not only the subgroup 57b of the lamp 50 that emits UV light in the area of the detected object 62, but also the corresponding subgroup 157b of the second lamp 150, may be switched off. Thus, as is directly apparent from the drawing, only a relatively small area is not illuminated with UV-C radiation so that fairly large gaps in the barrier may be prevented.

In the example in FIG. 18, illustrated strictly schematically, the lamps 50 and 150 have their own controllers 16 and 116, respectively. In the case that a joint sensor system 14 is to be used to control both lamps 50 and 150, communication between the controller 16 or the sensor system 14 of the lamp 50 and the controller 116 of the lamp 150 is provided. Alternatively, as shown in FIG. 1, an external controller for controlling the illuminants in a plurality of lamps 50, 150, etc., may also be provided.

The above statements with regard to FIG. 18 correspondingly apply when a subgroup includes an assembly or multiple assemblies.

If multiple groups are situated symmetrically with respect to the plane of symmetry S, the subdivision of the illuminants is preferably identical for both subgroups. No safety device is then necessary between the symmetrically situated groups. It is sufficient to provide such a safety device on the respective outwardly directed sides. The mutually corresponding subgroups or assemblies of the two groups are jointly controlled. Corresponding subgroups or assemblies are defined by identical positions with respect to the x axis, and identical sizes.

FIG. 19 shows a further embodiment of the invention, including a stand 20 for the radiation sources 10 of a system according to the invention, which radiate UV-C radiation in a horizontal direction in order to form UV-C walls 10b. Depending on the application, a stand 20 may be equipped with one, two, three, or four radiation sources 10 which, starting from the stand 20, may generate up to four UV-C walls 10b radiating in different spatial directions. The radiated UV-C light may be absorbed by neighboring stands, or by absorber walls or absorber stands set up for this purpose.

In further embodiments of the invention not illustrated, the stands may hold light strips or radiation sources that radiate vertically downwardly. In addition, it is conceivable for the light strips or radiation sources to be situated on the floor and to radiate toward the ceiling.

1. A lamp for forming a barrier for pathogens in indoor air, comprising a plurality of UV-C radiation-emitting illuminants (51, 51a, 51b; 151.1, 151.2) and a plurality of optical elements (54, 54a, 54b; 154 U, 154 O) for collimating radiation, which in each case are associated with an illuminant (51, 51a, 51b; 151.1, 151.2), the plurality of illuminants (51, 51a, 51b; 151.1, 151.2) and these associated optical elements (54, 54a, 54b; 154 U, 154 O) forming at least one group, and the radiation directions R of the collimated radiation that is emitted by the illuminants (51, 51a, 51b; 151.1, 151.2) within a group being situated in a shared surface, in particular a plane.

2. The lamp according to claim 1, characterized in that the radiation directions R of the collimated radiation that is emitted by the illuminants (51, 51a, 51b) within a group are parallel to one another, or the radiation directions R of assemblies within a group are parallel to one another, wherein an assembly includes multiple illuminants (151.1, 151.2) of a group together with their associated optical elements (154 U, 154 O).

3. The lamp according to claim 1 or 2, characterized in that each group includes multiple assemblies.

4. The lamp according to claim 1, characterized in that the optical device (54, 55, 154) includes a screening device (55) for screening out divergent radiation components.

5. The lamp according to one of claims 1 through 3, characterized in that the illuminants are LEDs (154.1, 154.2).

6. The lamp according to claim 4, characterized in that each illuminant (154.1, 154.2) is made up of at least two LED chips (176.1, 176.2) that are arranged in succession in the longitudinal direction of the lamp (50).

7. The lamp according to one of claims 1 through 5, characterized in that the illuminants (51, 51a, 51b) of at least one group are divided into subgroups (57a, 57b, 57c; 157a, 157b, 157c), and the illuminants (51, 51a, 51b) of these subgroups (57a, 57b, 57c; 157a, 157b, 157c) may be jointly switched on and off, but independently of the illuminants (51, 51a, 51b) of other subgroups (57a, 57b, 57c; 157a, 157b, 157c).

8. The lamp according to claim 7, characterized in that each subgroup corresponds to an assembly.

9. The lamp according to one of claims 1 through 6, characterized in that the lamp includes two groups that are situated symmetrically with respect to a center plane of the lamp.

10. The lamp according to one of claims 1 through 7, characterized in that the lamp (10, 50, 150) is designed as a light strip for ceiling or wall mounting.

11. The lamp according to one of claims 1 through 8, characterized in that the pathogen-deactivating UV-C radiation that is bundled to form a UV-C wall is far UV-C radiation having a wavelength in the range of 200-222 nm, in particular 207-222 nm.

12. The lamp according to one of claims 1 through 9, characterized in that the pathogen-deactivating UV-C radiation that is bundled to form a UV-C wall is UV-C radiation having a wavelength in the range of 223-280 nm, in particular having a wavelength greater than 242 nm.

13. A system for preventing or minimizing the spread of pathogens in indoor air, including one or more radiation sources (10) in the form of one or more lamps (10, 50, 150) according to one of claims 1 through 10, characterized in that the system comprises a sensor system (14) for detecting a penetration of one or more persons (P) or objects into a safety zone that is formed adjacent to the radiation field, and a controller (16) that is designed to at least partially switch the one or more radiation sources (10, 50, 150) on or off as a function of at least the presence of the person(s) (P) and/or objects, the controller (16) being designed to at least partially switch off the radiation source (10, 50, 150) in question when the sensor system (14) detects a penetration.

14. The system according to claim 11, characterized by freely movable stands (20) for holding one or more radiation sources (10, 50, 150).

15. The system according to one of claim 11 or 12, characterized in that the one or more radiation sources (10, 50, 150) are designed for arrangement along boundaries of room segments (12), the controller (16) being designed to activate the radiation sources (10, 50, 150) in question when one or more persons (P) are present in the room segment (12) in question, and to deactivate at least one of the radiation sources (10, 50, 150) when a person (P) enters or leaves the room segment (12).

16. The system according to one of claim 13 or 15, characterized in that further radiation sources (18) having a pathogen-deactivating or disinfecting effect are situated within the room segments (12), and the controller (16) is designed to activate the further radiation sources (18) when no person (P) is present in the room segment (12).

17. The system according to one of claims 13 through 16, characterized in that the sensor system includes a 3D camera or TOF camera and/or one or more CCD cameras.

18. The system according to one of claims 13 through 17, characterized in that the sensor system (14) includes at least one light source, and is configured to detect changes in the reflected portion of the light that is emitted by the light source and reflected from objects (62) in the surroundings.

19. The system according to one of claims 13 through 18, characterized in that the sensor system (14) is configured to determine a penetration into the safety zone with spatial resolution, and the controller (16) is configured to switch off at least one illuminant (51, 51a, 51b) based on the location of the penetration.

20. A method for preventing or minimizing the spread of viruses in indoor air, using one or more radiation sources (10) in a room, characterized in that the method includes generating at least one radiation field (10b), using at least one lamp according to one of claims 1 through 12, and detecting a movement or a presence of one or more persons (P) or objects (62) in the room, and automatically switching at least a portion of the illuminants of one or more radiation sources (10) on or off as a function of at least the presence of the person (P) or object (62).
